(19) United States
(12) Patent Application Publication
(10) Pub. No.: US 2023/0084196 A1
(43) Pub. Date: Mar. 16, 2023

(54) NMASS SPECTROMETRY-BASED STRATEGY FOR CHARACTERIZING HIGH MOLECULAR WEIGHT SPECIES OF A BIOLOGIC

(71) Applicant: Regeneron Pharmaceuticals, Inc., Tarrytown, NY (US)

(72) Inventors: Yuetian Yan, Chappaqua, NY (US); Shunhai Wang, Scarsdale, NY (US)

(21) Appl. No.: 17/943,776

(22) Filed: Sep. 13, 2022

Related U.S. Application Data
(60) Provisional application No. 63/243,835, filed on Sep. 14, 2021.

(51) Int. Cl. G01N 33/68; G01N 1/28; G01N 30/72

(52) U.S. Cl. CPC G01N 33/6848; G01N 1/28; G01N 30/7266; G01N 2030/8831

(57) ABSTRACT

The present invention relates to the field of protein characterization, and in particular to methods for characterizing high molecular weight species of a therapeutic protein by implementing a workflow including using a post-column denaturation-assisted SEC-MS method that allows highly specific, sensitive, and comprehensive characterization of high molecular weight species.

### CROSS- REFERENCE TO RELATED APPLICATIONS

This application claims priority to and the benefit of U.S. Provisional Patent Application No. 63/243,835, filed Sep. 14, 2021, which is herein incorporated by reference.

### FIELD

The present invention generally pertains to methods for characterizing high molecular weight size variants of a therapeutic protein using a size exclusion chromatography-mass spectrometry workflow.

### BACKGROUND

Therapeutic proteins have emerged as important drugs for the treatment of cancer, autoimmune disease, infection and cardiometabolic disorders, and they represent one of the fastest growing product segments of the pharmaceutical industry. Therapeutic protein products must meet very high standards of purity. Thus, it can be important to monitor impurities at different stages of drug development, production, storage and handling of therapeutic proteins.

The high molecular weight (HMW) size variants can be present as impurities in therapeutic protein samples and need to be closely monitored and characterized due to their impact on product safety and efficacy. Because of the complexity and often low abundances of HMW size variants in final drug substance (DS) samples, characterization of such HMW species is challenging and traditionally requires offline enrichment of the HMW species followed by analysis using various analytical tools.

Thus, there is a long felt need in the art for an efficient method for characterizing such HMW species in therapeutic protein products.

### SUMMARY

Exemplary embodiments disclosed herein satisfy the aforementioned demands by providing methods for characterizing such HMW species in therapeutic protein product by using a post-column denaturation-assisted native size exchange chromatography coupled online with a mass spectrometer (SEC-MS) method. This can allow highly specific, sensitive, and comprehensive characterization of HMW species directly from unfractionated samples. This method not only provides high-confidence identification of HMW species based on accurate mass measurement of both the intact assembly and the constituent subunits but also allows in-depth analysis of the interaction nature and location. In addition, using the extracted ion chromatograms, derived from high-quality, native-like mass spectra, the elution profiles of each non-covalent and/or non-dissociable complex can be readily reconstructed, facilitating the comprehension of a complex HMW profile. As this method does not require prior enrichment, it is thus desirable for providing both rapid and in-depth characterization of HMW species during the development of therapeutic protein products.

This disclosure provides a method for characterizing at least one high molecular weight species of a protein of interest, said method comprising obtaining a sample including said protein of interest and said at least one high molecular weight species; contacting said sample to a size exclusion chromatography column; washing said column to collect an eluate; adding a denaturing solution to the eluate to form a mixture; and subjecting said mixture to a mass spectrometer to characterize said at least one high molecular weight species.

In one aspect of this embodiment, the protein of interest is an antibody, a bispecific antibody, a multispecific antibody, antibody fragment, monoclonal antibody, or an Fc fusion protein.

In one aspect of this embodiment, said eluate includes said at least one high molecular weight species. In one aspect of this embodiment, said mixture is also subjected to ultraviolet detection.

In one aspect of this embodiment, the mass spectrometer is an electrospray ionization mass spectrometer. In a specific aspect of this embodiment, the mass spectrometer is a nano-electrospray ionization mass spectrometer

In one aspect of this embodiment, said mass spectrometer is operated under native conditions. In a specific aspect, the method further comprises comparing at least one peak from a mass spectra obtained using with a mass spectra obtained by carrying out an online size-exclusion chromatography-mass spectrometry of said sample under native conditions.

In one aspect of this embodiment, said denaturing solution comprises acetonitrile, formic acid, or combination of acetonitrile and formic acid. In a specific aspect of this embodiment, said denaturing solution comprises about 60% v/v acetonitrile and 4% v/v formic acid. In another specific aspect of this embodiment, said denaturing solution comprises about 60% v/v acetonitrile.

In one aspect of this embodiment, said mass spectrometer is operated under native conditions.

In one aspect of this embodiment, a flow of said mixture in said mass spectrometer is less than about 10 μL/min.

In one aspect of this embodiment, said mixture is split into said mass spectrometer and ultraviolet detector. In a specific aspect of this embodiment, a multi-nozzle emitter is used to add a desolvation gas with said mixture.

In one aspect of this embodiment, a desolvation gas is added to said mixture of (d) prior to subjecting it to mass spectrometer.

In one aspect of this embodiment, said at least one high molecular weight species is a non-covalent high molecular weight species of said protein of interest or a non-dissociable high molecular weight species of said protein of interest.

In one aspect of this embodiment, the method further comprises comparing at least one peak from a mass spectra with a mass spectra obtained by carrying out an online size-exclusion chromatography-mass spectrometry of said sample.

This disclosure also provides a method for characterizing at least one high molecular weight species of a protein of interest, said method comprising obtaining a sample including said protein of interest and said at least one high molecular weight species; digesting said sample using a hydrolyzing agent to form a digested sample; contacting said digested sample to a size exclusion chromatography column; washing said column to collect an eluate; adding a denaturing solution to the eluate to form a mixture; and subjecting said mixture to a mass spectrometer to characterize said at least one high molecular weight species.

In one aspect of this embodiment, the protein of interest is an antibody, a bispecific antibody, a multispecific antibody, antibody fragment, monoclonal antibody, or an Fc fusion protein.

In one aspect of this embodiment, said eluate includes said at least one high molecular weight species. In one aspect of this embodiment, said mixture is also subjected to ultraviolet detection.

In one aspect of this embodiment, the mass spectrometer is an electrospray ionization mass spectrometer. In a specific aspect of this embodiment, the mass spectrometer is a nano-electrospray ionization mass spectrometer

In one aspect of this embodiment, said mass spectrometer is operated under native conditions. In a specific aspect, the method further comprises comparing at least one peak from a mass spectra with a mass spectra obtained by carrying out an online size-exclusion chromatography-mass spectrometry of said sample under native conditions.

In one aspect of this embodiment, said denaturing solution comprises acetonitrile, formic acid, or combination of acetonitrile and formic acid. In a specific aspect of this embodiment, said denaturing solution comprises about 60% v/v acetonitrile and 4% v/v formic acid. In another specific aspect of this embodiment, said denaturing solution comprises about 60% v/v acetonitrile.

In one aspect of this embodiment, said mass spectrometer is operated under native conditions.

In one aspect of this embodiment, a flow of said mixture in said mass spectrometer is less than about 10 μL/min.

In one aspect of this embodiment, said mixture is split into said mass spectrometer and ultraviolet detector. In a specific aspect of this embodiment, a multi-nozzle emitter is used to add said desolvation gas with said mixture.

In one aspect of this embodiment, a desolvation gas is added to said mixture prior to subjecting it to mass spectrometer.

In one aspect of this embodiment, said at least one high molecular weight species is a non-covalent high molecular weight species of said protein of interest or a non-dissociable high molecular weight species of said protein of interest.

In one aspect of this embodiment, the method further comprises comparing at least one peak from a mass spectra with a mass spectra obtained by carrying out an online size-exclusion chromatography-mass spectrometry of said sample.

This disclosure also provides a method for characterizing at least one high molecular weight species, said method comprising obtaining a sample including at least two proteins of interest and said at least one high molecular weight species; contacting said sample to a size exclusion chromatography column; washing said column to collect an eluate; adding a denaturing solution to the eluate to form a mixture; and subjecting said mixture to a mass spectrometer to characterize said at least one high molecular weight species.

In one aspect of this embodiment, the protein of interest is an antibody, a bispecific antibody, a multispecific antibody, antibody fragment, monoclonal antibody, or an Fc fusion protein.

In one aspect of this embodiment, said eluate includes said at least one high molecular weight species. In one aspect of this embodiment, said mixture is also subjected to ultraviolet detection.

In one aspect of this embodiment, the mass spectrometer is an electrospray ionization mass spectrometer. In a specific aspect of this embodiment, the mass spectrometer is a nano-electrospray ionization mass spectrometer

In one aspect of this embodiment, said mass spectrometer is operated under native conditions. In a specific aspect, the method further comprises comparing at least one peak from a mass spectra obtained using with a mass spectra obtained by carrying out an online size-exclusion chromatography-mass spectrometry of said sample under native conditions.

In one aspect of this embodiment, said denaturing solution comprises acetonitrile, formic acid, or combination of acetonitrile and formic acid. In a specific aspect of this embodiment, said denaturing solution comprises about 60% v/v acetonitrile and 4% v/v formic acid. In another specific aspect of this embodiment, said denaturing solution comprises about 60% v/v acetonitrile.

In one aspect of this embodiment, said mass spectrometer is operated under native conditions.

In one aspect of this embodiment, a flow of said mixture in said mass spectrometer is less than about 10 μL/min.

In one aspect of this embodiment, said mixture is split into said mass spectrometer and ultraviolet detector. In a specific aspect of this embodiment, a multi-nozzle emitter is used to add said desolvation gas with said mixture.

In one aspect of this embodiment, a desolvation gas is added to said mixture of (d) prior to subjecting it to mass spectrometer.

In one aspect of this embodiment, said at least one high molecular weight species is a non-covalent high molecular weight species of said protein of interest or a non-dissociable high molecular weight species of said protein of interest.

In one aspect of this embodiment, the method further comprises comparing at least one peak from a mass spectra with a mass spectra obtained by carrying out an online size-exclusion chromatography-mass spectrometry of said sample.

In one aspect of this embodiment, said sample is digested using a hydrolyzing agent prior to subjecting it to size-exclusion chromatography column. In a specific aspect, the hydrolyzing agent is immunoglobulin-degrading enzyme of Streptococcus pyogenes (IdeS) or its variant.

### BRIEF DESCRIPTION OF THE DRAWINGS

FIG. 1 displays the effectiveness of the present invention using an exemplary embodiment.

FIG. 2 show relative amount impurities generally present in a therapeutic protein products.

FIG. 3 is a representation of the present invention according to an exemplary embodiment.

FIG. 4A shows a mass spectra of partially reduced mAb1 (inter-chain disulfide bonds disrupted) obtained under native (black trace) or PCD conditions (orange and red traces) obtained according to an exemplary embodiment.

FIG. 4B shows a mass spectra of mAb2 dimer obtained under native (black trace) or PCD (orange and red traces) conditions obtained according to an exemplary embodiment.

FIG. 5 shows a nSEC-UV/MS analysis of mAb3 enriched HMW sample after IdeS digestion displaying the SEC-UV trace (central panel), peak assignment, and the deconvoluted mass spectra for each HMW peak obtained under native (blue traces) or PCD (red traces) conditions, according to an exemplary embodiment.

FIG. 6 shows a tabulated summary of size variant masses associated with FabRICATOR-digested and deglycosylated enriched mAb3 HMW sample, according to an exemplary embodiment.

FIG. 7 shows a nSEC-UV/MS analysis of bsAb DS sample displaying the SEC-TICs (left panel, red and blue traces) and the raw mass spectra for each HMW peak obtained under native (blue traces) or PCD (red traces) conditions, according to an exemplary embodiment. The XICs were generated using the most abundant charge state of each species (grey traces, left panels).

FIG. 8 shows a tabulated summary of size variant masses associated with deglycosylated bsAb sample, according to an exemplary embodiment.

FIG. 9 shows HMW profiles of mAb4 DS lot 1 and lot 2 characterized at a) intact level and b) subdomain level (after IdeS digestion) using PCD-assisted nSEC-UV/MS analysis. The UV profile (black trace) and XICs (colored traces) representing the elution profile of each HMW-related species were shown (only HMW region displayed), according to an exemplary embodiment. The XICs were generated using the most abundant charge state of each species.

FIG. 10 shows a tabulated summary of non-dissociable dimeric species detected in mAb4 lot 1 and lot 2 DS samples by PCD-assisted nSEC-MS at intact level and sub-domain level, according to an exemplary embodiment.

FIG. 11 shows HMW species detected in co-formulated mAb-A and mAb-B samples at a) T0 and b) 25° C. for 6 months using nSEC-MS under both native (black trace) and PCD (red trace) conditions, according to an exemplary embodiment. The relative abundance of each dimer was estimated using the integrated peak areas from the deconvoluted mass spectra and annotated.

FIG. 12 shows a native SEC-UV traces of co-formulated mAb-A and mAb-B at T0 and after stored under 25° C. for 6 months, according to an exemplary embodiment.

### DETAILED DESCRIPTION

Identification and quantification of product-related variants in biologic products can be very important during the production and development of a product. The identification of such variants can be imperative into developing a safe and effective product. Hence, a robust method and/or workflow to characterize such variants can be beneficial.

Therapeutic proteins often exhibit some degree of size heterogeneity containing product-related impurities, including HMW aggregates and low molecular weight (LMW) fragments. These species often arise from chemical and enzymatic degradation of the mAb molecules due to environmental stresses during product manufacture, shipping, and storage (Roberts C J. Therapeutic protein aggregation: Mechanisms, design, and control. Trends Biotechnol 2014: 32(7): 372-380; Cordoba A J, Shyong B J, Breen D, Harris R J. Non-enzymatic hinge region fragmentation of antibodies in solution. J Chromatogr B Analyt Technol Biomed Life Sci 2005: 818(2): 115-121; Xiang T, Lundell E, Sun Z, Liu H. Structural effect of a recombinant monoclonal antibody on hinge region peptide bond hydrolysis. J Chromatogr B Analyt Technol Biomed Life Sci 2007: 858(1-2): 254-262). LMW fragments can be generated via different chemical or enzymatic degradation pathways (e.g., acid-, base- and enzyme-driven hydrolysis of polypeptide bonds inter-chain disulfide bond breakage, etc.), yielding truncated forms of the mAb molecule (Wang S, Liu AP, Yan Y, Daly TJ, Li N. Characterization of product-related low molecular weight impurities in therapeutic monoclonal antibodies using hydrophilic interaction chromatography coupled with mass spectrometry. J Pharm Biomed Anal 2018: 154(468-475; Vlasak J, Ionescu R. Fragmentation of monoclonal antibodies. MAbs 2011: 3(3): 253-263).

In contrast, the formation of HMW species is a much more complex process. The generated HMW forms can vary in size, conformation, interaction nature (covalent or non-covalent), and site of association (Paul R, Graff-Meyer A, Stahlberg H, Lauer M E, Rufer A C, Beck H, Briguet A, Schnaible V, Buckel T, Boeckle S. Structure and function of purified monoclonal antibody dimers induced by different stress conditions. Pharm Res 2012: 29(8): 2047-2059). Besides the stress conditions, the protein primary sequence, as well as its higher-order structure, all contribute to its tendency to aggregation via different pathways. Therefore, it is nearly impossible to use a general rule to predict or describe the protein aggregation behavior of each molecule. As HMW species (from soluble oligomers to visible particles) may impact drug safety and efficacy by eliciting unwanted immunogenic responses and/or altering its pharmacokinetic behaviors (Narhi L O, Schmit J, Bechtold-Peters K, Sharma D. Classification of protein aggregates. J Pharm Sci 2012: 101(2): 493-498), detailed characterization, continuous monitoring and control of the HMW species throughout the product life cycle are required (Parenky A, Myler H, Amaravadi L, Bechtold-Peters K, Rosenberg A, Kirshner S, Quarmby V. New FDA draft guidance on immunogenicity. AAPS J 2014: 16(3): 499-503). In addition, deep understanding of the aggregation mechanisms, as achieved by in-depth characterization, not only provides the framework for risk assessment of HMW species, but might also offer insights for designing protein molecules with reduced aggregation propensity through protein engineering.

Characterization of HMW size variants in therapeutic protein products often relies on an arsenal of analytical and biophysical tools due to their complexity. Both sedimentation velocity analytical ultra-centrifugation (SV-AUC) and size exclusion chromatography (SEC) have been widely used in characterizing mAb HMW species due to their excellent resolving power and quantitative performance (Lebowitz J, Lewis M S, Schuck P. Modern analytical ultracentrifugation in protein science: A tutorial review. Protein Sci 2002: 11(9): 2067-2079; Hughes H, Morgan C, Brunyak E, Barranco K, Cohen E, Edmunds T, Lee K. A multi-tiered analytical approach for the analysis and quantitation of high-molecular-weight aggregates in a recombinant therapeutic glycoprotein. AAPS J 2009: 11(2): 335-341). In particular, SEC with UV detection is routinely used as a batch release assay to directly monitor the level and elution profile of soluble aggregates in therapeutic mAb products (Lowe D, Dudgeon K, Rouet R, Schofield P, Jermutus L, Christ D. Aggregation, stability, and formulation of human antibody therapeutics. Adv Protein Chem Struct Biol 2011: 8441-61; Zolls S, Tantipolphan R, Wiggenhorn M, Winter G, Jiskoot W, Friess W, Hawe A. Particles in therapeutic protein formulations, part 1: Overview of analytical methods. J Pharm Sci 2012: 101(3): 914-935). To enable detailed elucidation of the HMW species and gain insights on aggregation mechanisms, enrichment of the mAb HMW species followed by in-depth characterization by other techniques is almost always required (Paul R. et al., supra; Rouby G, Tran N T, Leblanc Y, Taverna M, Bihoreau N. Investigation of monoclonal antibody dimers in a final formulated drug by separation techniques coupled to native mass spectrometry. MAbs 2020: 12(1): e1781743; Lu C, Liu D, Liu H, Motchnik P. Characterization of monoclonal antibody size variants containing extra light chains. MAbs 2013: 5(1): 102-113; Remmele R L, Jr., Callahan W J, Krishnan S, Zhou L, Bondarenko P V, Nichols A C, Kleemann G R, Pipes G D, Park S, Fodor S et al. Active dimer of epratuzumab provides insight into the complex nature of an antibody aggregate. J Pharm Sci 2006: 95(1): 126-145; Iwura T, Fukuda J, Yamazaki K, Kanamaru S, Arisaka F. Intermolecular interactions and conformation of antibody dimers present in igg1 biopharmaceuticals. J Biochem 2014: 155(1): 63-71; Plath F, Ringler P, Graff-Meyer A, Stahlberg H, Lauer ME, Rufer A C, Graewert M A, Svergun D, Gellermann G, Finkler C et al. Characterization of mab dimers reveals predominant dimer forms common in therapeutic mabs. MAbs 2016: 8(5): 928-940). For example, capillary electrophoresis-sodium dodecyl sulfate (CE-SDS) performed under non-reducing conditions can be used to differentiate and estimate the levels of covalently and non-covalently bound HMW species (Rouby G. et al., supra; Remmele R L et al., supra; Plath F. et al., supra). Moreover, when operated under reducing conditions, CE-SDS can further evaluate the possible contribution from intermolecular disulfide bond scrambling to the formation of covalent aggregates. Limited enzymatic digestion (e.g., IdeS digestion and limited Lys-C digestion) followed by mass spectrometry (MS) analysis has also proven effective in determining the aggregation interfaces at subdomain levels based on accurate mass measurement (Rouby G. et al., supra; Remmele R L et al., supra; Iwura et al., supra; Plath F. et al., supra). Finally, to achieve peptide-level or even residue-level elucidation of the aggregation interfaces and mechanisms, more sophisticated strategies, such as protein footprinting (e.g. hydrogen-deuterium exchange MS and hydroxyl radical footprinting) and bottom-up-based crosslinking analyses, can be applied to study the non-covalent and covalent HMW species, respectively (Iacob R E, Bou-Assaf G M, Makowski L, Engen J R, Berkowitz S A, Houde D. Investigating monoclonal antibody aggregation using a combination of h/dx-ms and other biophysical measurements. J Pharm Sci 2013: 102(12): 4315-4329; Zhang A, Singh S K, Shirts M R, Kumar S, Fernandez E J. Distinct aggregation mechanisms of monoclonal antibody under thermal and freeze-thaw stresses revealed by hydrogen exchange. Pharm Res 2012: 29(1): 236-250; Yan Y, Wei H, Jusuf S, Krystek S R, Jr., Chen J, Chen G, Ludwig R T, Tao L, Das T K. Mapping the binding interface in a noncovalent size variant of a monoclonal antibody using native mass spectrometry, hydrogen-deuterium exchange mass spectrometry, and computational analysis. J Pharm Sci 2017: 106(11): 3222-3229; Deperalta G, Alvarez M, Bechtel C, Dong K, McDonald R, Ling V. Structural analysis of a therapeutic monoclonal antibody dimer by hydroxyl radical footprinting. MAbs 2013: 5(1): 86-101).

Online coupling of SEC with direct MS detection under near native conditions (native SEC-MS) has gained a lot of interest over the past few years to study mAb BMW species (Rouby et al., supra; Ehkirch A, Hernandez-Alba O, Colas O, Beck A, Guillarme D, Cianferani S. Hyphenation of size exclusion chromatography to native ion mobility mass spectrometry for the analytical characterization of therapeutic antibodies and related products. J Chromatogr B Analyt Technol Biomed Life Sci 2018: 1086 (176-183); Haberger M, Leiss M, Heidenreich A K, Pester 0, Hafenmair G, Hook M, Bonnington L, Wegele H, Haindl M, Reusch D et al. Rapid characterization of biotherapeutic proteins by size-exclusion chromatography coupled to native mass spectrometry. MAbs 2016: 8(2): 331-339.

Using MS-compatible mobile phases that can preserve protein conformation and non-covalent interactions, native SEC-MS (nSEC-MS) can provide rapid and improved identification of size variants based on accurate mass measurement. In addition, thanks to the recent advances in both methodology and instrumentation, nSEC-MS has become a highly sensitive method that can readily detect very low levels of BMW species (e.g., at 0.01%) directly from unfractionated drug substance (DS) samples (Yan Y, Xing T, Wang S, Li N. Versatile, sensitive, and robust native 1c-ms platform for intact mass analysis of protein drugs. J Am Soc Mass Spectrom 2020: 31(10): 2171-2179). Despite these notable successes, application of the nSEC-MS method alone still cannot obtain a complete profile of the HMW species. First, as a non-denaturing method, nSEC-MS analysis does not distinguish between the non-covalently and covalently bound HMW complexes, unless clear mass differences resulting from the covalent crosslinks, can be detected. Unfortunately, the latter can be extremely difficult to achieve, due to both insufficient chromatographical resolution and mass resolving power for large complexes. For instance, dimer species formed by different mechanisms (e.g., non-covalent and covalent interactions) are often co-eluting during SEC separation and measured with an averaged mass by MS detection. Therefore, the distribution of non-covalent and covalent dimer species cannot be directly determined by nSEC-MS method. Second, compared to well-expected oligomeric species (e.g., dimer, trimer, tetramer, etc.), confident identification of unconventional HMW species (e.g., mAb monomer complexed with additional light chains) often cannot be established by intact mass measurement alone (Lu et al., supra; Yan et al., supra). This is because reduced mass accuracy is often expected for mass measurement of large BMW species present at low abundances, which can lead to ambiguous mass assignments.

To overcome these challenges, the present invention provides a new post-column denaturation-assisted nSEC-MS method (PCD-assisted nSEC-MS) that is optimized to dissociate SEC-resolved, non-covalent BMW species into constituent components for subsequent MS detection. As a result, this new approach enables simultaneous detection of both non-covalent and non-dissociable HMW species under identical SEC separation conditions. In addition, this strategy improves the identification of heterogeneous BMW species by 1) confirming the identities of the constituent subunits dissociated from the non-covalent HMW complexes; and 2) achieving more accurate mass measurement of non-dissociable BMW species by removing interference from co-eluting, non-covalent species. Furthermore, by incorporating a limited enzymatic digestion step, the PCD-assisted nSEC-MS method can readily reveal both the interaction nature and interaction interfaces of mAb aggregates at subdomain levels.

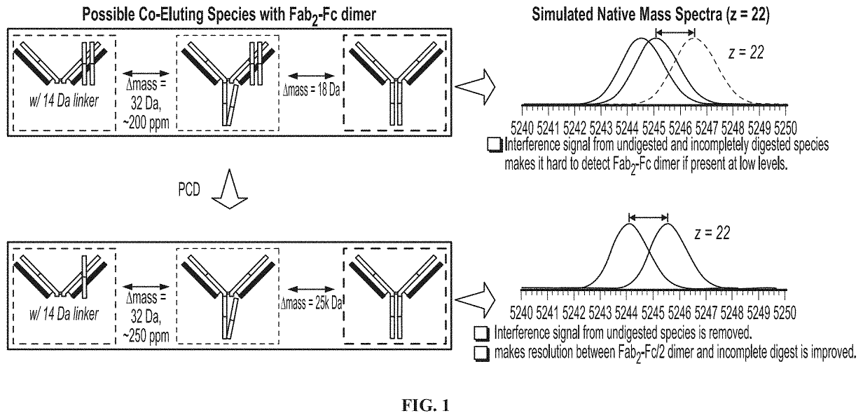

The present invention also provide a more accurate measurement of covalent crosslinks by (a) reducing the interference from co-eluting non-covalent species and (b) reducing the size of the species. For example, a co-eluting species with a Fab2-Fc dimer can create an interference due to undigested and partially digested species. See FIG. 1, top panel. Using the present invention, the interference signal from the undigested species can be removed by using a protease such as IdeS. See FIG. 1, bottom panel.

Unless described otherwise, all technical and scientific terms used herein have the same meaning as commonly understood by one of ordinary skill in the art to which this invention belongs. Although any methods and materials similar or equivalent to those described herein can be used in the practice or testing, particular methods and materials are now described. All publications mentioned are hereby incorporated by reference.

The term “a” should be understood to mean “at least one”; and the terms “about” and “approximately” should be understood to permit standard variation as would be understood by those of ordinary skill in the art; and where ranges are provided, endpoints are included.

In some exemplary embodiments, the disclosure provides a method for characterizing at least one high molecular weight species of a protein of interest.

As used herein, the term “protein,” “therapeutic protein,” or “protein of interest” includes any amino acid polymer having covalently linked amide bonds. Proteins comprise one or more amino acid polymer chains, generally known in the art as “polypeptides.” “Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof “Synthetic peptides or polypeptides' refers to a non-naturally occurring peptide or polypeptide. Synthetic peptides or polypeptides can be synthesized, for example, using an automated polypeptide synthesizer. Various solid phase peptide synthesis methods are known. A protein may contain one or multiple polypeptides to form a single functioning biomolecule. A protein can include any of bio-therapeutic proteins, recombinant proteins used in research or therapy, trap proteins and other chimeric receptor Fc-fusion proteins, chimeric proteins, antibodies, monoclonal antibodies, polyclonal antibodies, human antibodies, and bispecific antibodies. In another exemplary aspect, a protein can include antibody fragments, nanobodies, recombinant antibody chimeras, cytokines, chemokines, peptide hormones, and the like. Proteins may be produced using recombinant cell-based production systems, such as the insect bacculovirus system, yeast systems (e.g., Pichia sp.), mammalian systems (e.g., CHO cells and CHO derivatives like CHO-K1 cells). For a review discussing biotherapeutic proteins and their production, see Ghaderi et al., “Production platforms for biotherapeutic glycoproteins. Occurrence, impact, and challenges of non-human sialylation,” (BIOTECHNOL. GENET. ENG. REV. 147-175 (2012)). In some exemplary embodiments, proteins comprise modifications, adducts, and other covalently linked moieties. Those modifications, adducts and moieties include for example avidin, streptavidin, biotin, glycans (e.g., N-acetylgalactosamine, galactose, neuraminic acid, N-acetylglucosamine, fucose, mannose, and other monosaccharides), PEG, polyhistidine, FLAGtag, maltose binding protein (MBP), chitin binding protein (CBP), glutathione-S-transferase (GST) myc-epitope, fluorescent labels and other dyes, and the like. Proteins can be classified on the basis of compositions and solubility and can thus include simple proteins, such as, globular proteins and fibrous proteins; conjugated proteins, such as nucleoproteins, glycoproteins, mucoproteins, chromoproteins, phosphoproteins, metalloproteins, and lipoproteins; and derived proteins, such as primary derived proteins and secondary derived proteins.

In some exemplary embodiments, the protein can be an antibody, a bispecific antibody, a multispecific antibody, antibody fragment, monoclonal antibody, or an Fc fusion protein.

The term “antibody,” as used herein includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different exemplary embodiments, the FRs of the anti-big-ET-1 antibody (or antigen-binding portion thereof) may be identical to the human germline sequences, or may be naturally or artificially modified. An amino acid consensus sequence may be defined based on a side-by-side analysis of two or more CDRs. The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, for example, from full antibody molecules using any suitable standard techniques such as proteolytic digestion or recombinant genetic engineering techniques involving the manipulation and expression of DNA encoding antibody variable and optionally constant domains. Such DNA is known and/or is readily available from, for example, commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

As used herein, an “antibody fragment” includes a portion of an intact antibody, such as, for example, the antigen-binding or variable region of an antibody. Examples of antibody fragments include, but are not limited to, a Fab fragment, a Fab′ fragment, a F(ab′)2 fragment, a Fc fragment, a scFv fragment, a Fv fragment, a dsFv diabody, a dAb fragment, a Fd′ fragment, a Fd fragment, and an isolated complementarity determining region (CDR) region, as well as triabodies, tetrabodies, linear antibodies, single-chain antibody molecules, and multi specific antibodies formed from antibody fragments. Fv fragments are the combination of the variable regions of the immunoglobulin heavy and light chains, and ScFv proteins are recombinant single chain polypeptide molecules in which immunoglobulin light and heavy chain variable regions are connected by a peptide linker. An antibody fragment may be produced by various means. For example, an antibody fragment may be enzymatically or chemically produced by fragmentation of an intact antibody and/or it may be recombinantly produced from a gene encoding the partial antibody sequence. Alternatively or additionally, an antibody fragment may be wholly or partially synthetically produced. An antibody fragment may optionally comprise a single chain antibody fragment. Alternatively or additionally, an antibody fragment may comprise multiple chains that are linked together, for example, by disulfide linkages. An antibody fragment may optionally comprise a multi-molecular complex.

The term “monoclonal antibody” as used herein is not limited to antibodies produced through hybridoma technology. A monoclonal antibody can be derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof

The term “Fc fusion proteins” as used herein includes part or all of two or more proteins, one of which is an Fc portion of an immunoglobulin molecule, that are not fused in their natural state. Preparation of fusion proteins comprising certain heterologous polypeptides fused to various portions of antibody-derived polypeptides (including the Fc domain) has been described, e.g., by Ashkenazi et al., Proc. Natl. Acad. ScL USA 88: 10535, 1991; Byrn et al., Nature 344: 677, 1990; and Hollenbaugh et al., “Construction of Immunoglobulin Fusion Proteins”, in Current Protocols in Immunology, Suppl. 4, pages 10.19.1-10.19.11, 1992. “Receptor Fc fusion proteins” comprise one or more of one or more extracellular domain(s) of a receptor coupled to an Fc moiety, which in some embodiments comprises a hinge region followed by a CH2 and CH3 domain of an immunoglobulin. In some embodiments, the Fc-fusion protein contains two or more distinct receptor chains that bind to a single or more than one ligand(s). For example, an Fc-fusion protein is a trap, such as for example an IL-1 trap (e.g., Rilonacept, which contains the IL-1 RAcP ligand binding region fused to the IL-1R1 extracellular region fused to Fc of hIgGl; see U.S. Pat. No. 6,927,004, which is herein incorporated by reference in its entirety), or a VEGF Trap (e.g., Aflibercept, which contains the Ig domain 2 of the VEGF receptor Flt1 fused to the Ig domain 3 of the VEGF receptor Flk1 fused to Fc of hIgGl; e.g., SEQ ID NO: 1; see U.S. Pat. Nos. 7,087,411 and 7,279,159, which are herein incorporated by reference in their entirety).

As used herein, the term “impurity” can include any undesirable protein present in the protein biopharmaceutical product. Impurity can include process and product-related impurities. The impurity can further be of known structure, partially characterized, or unidentified.

Process-related impurities can be derived from the manufacturing process and can include the three major categories: cell substrate-derived, cell culture-derived and downstream derived. Cell substrate-derived impurities include, but are not limited to, proteins derived from the host organism and nucleic acid (host cell genomic, vector, or total DNA). Cell culture-derived impurities include, but are not limited to, inducers, antibiotics, serum, and other media components. Downstream-derived impurities include, but are not limited to, enzymes, chemical and biochemical processing reagents (e.g., cyanogen bromide, guanidine, oxidizing and reducing agents), inorganic salts (e.g., heavy metals, arsenic, nonmetallic ion), solvents, carriers, ligands (e.g., monoclonal antibodies), and other leachables.

Product-related impurities (e.g., precursors, certain degradation products) can be molecular variants arising during manufacture and/or storage that do not have properties comparable to those of the desired product with respect to activity, efficacy, and safety. Such variants may need considerable effort in isolation and characterization in order to identify the type of modification(s). Product-related impurities can include truncated forms, modified forms, and aggregates. Truncated forms are formed by hydrolytic enzymes or chemicals which catalyze the cleavage of peptide bonds. Modified forms include, but are not limited to, deamidated, isomerized, mismatched S-S linked, oxidized, or altered conjugated forms (e.g., glycosylation, phosphorylation). Modified forms can also include any post-translationally modified form. Aggregates include dimers and higher multiples of the desired product. (Q6B Specifications: Test Procedures and Acceptance Criteria for Biotechnological/Biological Products, ICH August 1999, U.S. Dept. of Health and Humans Services).

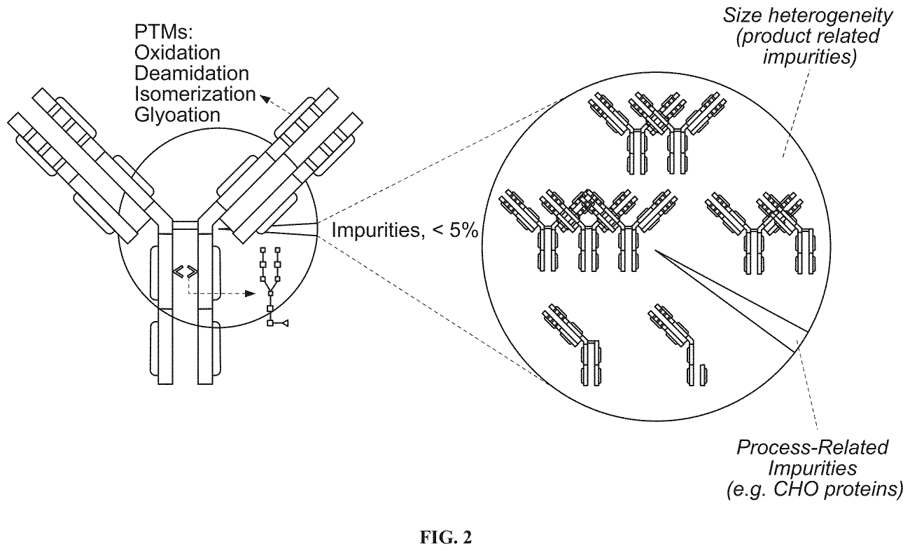

As shown in FIG. 2, product related impurities are the major impurities in therapeutic protein products and thus need careful characterization. Some product-related impurities or product-related protein variants have compromised binding affinity. Compromised binding affinity, here, includes a reduced binding affinity to the target of the protein of interest in the body or an antigen designed for the protein of interest. The compromised binding affinity can be any affinity which is less than the affinity of the protein of interest towards the target of the protein of interest in the body or an antigen designed for the protein of interest.

As used herein, the general term “post-translational modifications” or “PTMs” refers to covalent modifications that polypeptides undergo, either during (co-translational modification) or after (post-translational modification) their ribosomal synthesis. PTMs are generally introduced by specific enzymes or enzyme pathways. Many occur at the site of a specific characteristic protein sequence (signature sequence) within the protein backbone. Several hundred PTMs have been recorded, and these modifications invariably influence some aspect of a protein's structure or function (Walsh, G. “Proteins” (2014) second edition, published by Wiley and Sons, Ltd., ISBN: 9780470669853). The various post-translational modifications include, but are not limited to, cleavage, N-terminal extensions, protein degradation, acylation of the N-terminus, biotinylation (acylation of lysine residues with a biotin), amidation of the C-terminal, glycosylation, iodination, covalent attachment of prosthetic groups, acetylation (the addition of an acetyl group, usually at the N-terminus of the protein), alkylation (the addition of an alkyl group (e.g. methyl, ethyl, propyl) usually at lysine or arginine residues), methylation, adenylation, ADP-ribosylation, covalent cross links within, or between, polypeptide chains, sulfonation, prenylation, Vitamin C dependent modifications (proline and lysine hydroxylations and carboxy terminal amidation), Vitamin K dependent modification wherein Vitamin K is a cofactor in the carboxylation of glutamic acid residues resulting in the formation of a y-carboxyglutamate (a glu residue), glutamylation (covalent linkage of glutamic acid residues), glycylation (covalent linkage glycine residues), glycosylation (addition of a glycosyl group to either asparagine, hydroxylysine, serine, or threonine, resulting in a glycoprotein), isoprenylation (addition of an isoprenoid group such as farnesol and geranylgeraniol), lipoylation (attachment of a lipoate functionality), phosphopantetheinylation (addition of a 4′-phosphopantetheinyl moiety from coenzyme A, as in fatty acid, polyketide, non-ribosomal peptide and leucine biosynthesis), phosphorylation (addition of a phosphate group, usually to serine, tyrosine, threonine or histidine), and sulfation (addition of a sulfate group, usually to a tyrosine residue). The post-translational modifications that change the chemical nature of amino acids include, but are not limited to, citrullination (the conversion of arginine to citrulline by deimination), and deamidation (the conversion of glutamine to glutamic acid or asparagine to aspartic acid). The post-translational modifications that involve structural changes include, but are not limited to, formation of disulfide bridges (covalent linkage of two cysteine amino acids) and proteolytic cleavage (cleavage of a protein at a peptide bond). Certain post-translational modifications involve the addition of other proteins or peptides, such as ISGylation (covalent linkage to the ISG15 protein (Interferon-Stimulated Gene)), SUMOylation (covalent linkage to the SUMO protein (Small Ubiquitin-related MOdifier)) and ubiquitination (covalent linkage to the protein ubiquitin). See European Bioinformatics Institute Protein Information ResourceSlB Swiss Institute of Bioinformatics, EUROPEAN BIOINFORMATICS INSTITUTE DRS—DROSOMYCIN PRECURSOR—DROSOPHILA MELANOGASTER (FRUIT FLY)—DRS GENE & PROTEIN, http://www.uniprot.org/docs/ptmlist (last visited Jan. 15, 2019) for a more detailed controlled vocabulary of PTMs curated by UniProt.

As used herein, the term “chromatography” refers to a process in which a chemical mixture carried by a liquid or gas can be separated into components as a result of differential distribution of the chemical entities as they flow around or over a stationary liquid or solid phase. Non-limiting examples of chromatography include traditional reversed-phased (RP), ion exchange (IEX), mixed mode chromatography and normal phase chromatography (NP).

Size exclusion chromatography or gel filtration relies on the separation of components as a function of their molecular size. Separation depends on the amount of time that the substances spend in the porous stationary phase as compared to time in the fluid. The probability that a molecule will reside in a pore depends on the size of the molecule and the pore. In addition, the ability of a substance to permeate into pores is determined by the diffusion mobility of macromolecules which is higher for small macromolecules. Very large macromolecules may not penetrate the pores of the stationary phase at all; and for very small macromolecules the probability of penetration is close to unity. While components of larger molecular size move more quickly past the stationary phase, components of small molecular size have a longer path length through the pores of the stationary phase and are thus retained longer in the stationary phase.

The chromatographic material can comprise a size exclusion material wherein the size exclusion material is a resin or membrane. The matrix used for size exclusion is preferably an inert gel medium which can be a composite of cross-linked polysaccharides, for example, cross-linked agarose and/or dextran in the form of spherical beads. The degree of cross-linking determines the size of pores that are present in the swollen gel beads. Molecules greater than a certain size do not enter the gel beads and thus move through the chromatographic bed the fastest. Smaller molecules, such as detergent, protein, DNA and the like, which enter the gel beads to varying extent depending on their size and shape, are retarded in their passage through the bed. Molecules are thus generally eluted in the order of decreasing molecular size.

Porous chromatographic resins appropriate for size-exclusion chromatography of viruses may be made of dextrose, agarose, polyacrylamide, or silica which have different physic& characteristics. Polymer combinations can also be also used. Most commonly used are those under the tradename. “SEPHADEX” available from Amersham Biosciences. Other size exclusion supports from different materials of construction are also appropriate, for example Toyopearl 55F (polymethacrylate, from Tosoh Bioscience, Montgomery Pa.) and Bio-Gel P-30 Fine (BioRad Laboratories, Hercules, Calif.).

As used herein, the term “Mixed Mode Chromatography (MMC)” or “multimodal chromatography” includes a chromatographic method in which solutes interact with stationary phase through more than one interaction mode or mechanism. MMC can be used as an alternative or complementary tool to traditional reversed-phased (RP), ion exchange (IEX) and normal phase chromatography (NP). Unlike RP, NP and IEX chromatography, in which hydrophobic interaction, hydrophilic interaction and ionic interaction respectively are the dominant interaction modes, mixed-mode chromatography can employ a combination of two or more of these interaction modes. Mixed mode chromatography media can provide unique selectivity that cannot be reproduced by single mode chromatography. Mixed mode chromatography can also provide potential cost savings and operation flexibility compared to affinity based methods. The present invention can include using a mixed mode chromatography capable to performing size exclusion based separation.

In some exemplary embodiments, the mobile phase used to obtain said eluate from size exclusion chromatography can comprise a volatile salt. In some specific embodiments, the mobile phase can comprise ammonium acetate, ammonium bicarbonate, or ammonium formate, or combinations thereof.

As used herein, the term “mass spectrometer” includes a device capable of identifying specific molecular species and measuring their accurate masses. The term is meant to include any molecular detector into which a polypeptide or peptide may be eluted for detection and/or characterization. A mass spectrometer can include three major parts: the ion source, the mass analyzer, and the detector. The role of the ion source is to create gas phase ions. Analyte atoms, molecules, or clusters can be transferred into gas phase and ionized either concurrently (as in electrospray ionization). The choice of ion source depends heavily on the application.

In some exemplary embodiments, the electrospray ionization mass spectrometer can be a nano-electrospray ionization mass spectrometer.

The term “nanoelectrospray” or “nanospray” as used herein refers to electrospray ionization at a very low solvent flow rate, typically microliters or hundreds of nanoliters per minute of sample solution or lower, often without the use of an external solvent delivery. The electrospray infusion setup forming a nanoelectrospray can use a static nanoelectrospray emitter or a dynamic nanoelectrospray emitter. A static nanoelectrospray emitter performs a continuous analysis of small sample (analyte) solution volumes over an extended period of time. A dynamic nanoelectrospray emitter uses a capillary column and a solvent delivery system to perform chromatographic separations on mixtures prior to analysis by the mass spectrometer.

As used herein, the term “mass analyzer” includes a device that can separate species, that is, atoms, molecules, or clusters, according to their mass. Non-limiting examples of mass analyzers that could be employed for fast protein sequencing are time-of-flight (TOF), magnetic/electric sector, quadrupole mass filter (Q), quadrupole ion trap (QIT), orbitrap, Fourier transform ion cyclotron resonance (FTICR), and also the technique of accelerator mass spectrometry (AMS).

In some exemplary embodiments, the mobile phase used for the methods is compatible with the mass spectrometer.

In some exemplary embodiments, the sample can comprise about 10 μg to about 100 μg of the protein of interest.

In some exemplary embodiments, the flow rate in the electrospray ionization mass spectrometer can be about 10 nL/min to about 1000 μ/min.

In some exemplary embodiments, the electrospray ionization mass spectrometer can have a spray voltage of about 0.8 kV to about 5 kV.

In some exemplary embodiments, mass spectrometry can be performed under native conditions.

As used herein, the term “native conditions” or “native MS” or “native ESI- MS” can include a performing mass spectrometry under conditions that preserve no-covalent interactions in an analyte. For detailed review on native MS, refer to the review: Elisabetta Boeri Erba & Carlo Petosa, The emerging role of native mass spectrometry in characterizing the structure and dynamics of macromolecular complexes, 24 PROTEIN SCIENCE1176-1192 (2015); (Hao Zhang et al., Native mass spectrometry of photosynthetic pigment-protein complexes, 587 FEBS Letters 1012-1020 (2013)).

In some exemplary embodiments, the mass spectrometer can be a tandem mass spectrometer.

As used herein, the term “tandem mass spectrometry” includes a technique where structural information on sample molecules is obtained by using multiple stages of mass selection and mass separation. A prerequisite is that the sample molecules can be transferred into gas phase and ionized intact and that they can be induced to fall apart in some predictable and controllable fashion after the first mass selection step. Multistage MS/MS, or MSn, can be performed by first selecting and isolating a precursor ion (MS²), fragmenting it, isolating a primary fragment ion (MS³), fragmenting it, isolating a secondary fragment (MS⁴), and so on as long as one can obtain meaningful information or the fragment ion signal is detectable. Tandem MS have been successfully performed with a wide variety of analyzer combinations. What analyzers to combine for a certain application is determined by many different factors, such as sensitivity, selectivity, and speed, but also size, cost, and availability. The two major categories of tandem MS methods are tandem-in-space and tandem-in-time, but there are also hybrids where tandem-in-time analyzers are coupled in space or with tandem-in-space analyzers. A tandem-in-space mass spectrometer comprises an ion source, a precursor ion activation device, and at least two non-trapping mass analyzers. Specific m/z separation functions can be designed so that in one section of the instrument ions are selected, dissociated in an intermediate region, and the product ions are then transmitted to another analyzer for m/z separation and data acquisition. In tandem-in-time mass spectrometer ions produced in the ion source can be trapped, isolated, fragmented, and m/z separated in the same physical device.

The peptides identified by the mass spectrometer can be used as surrogate representatives of the intact protein and their post-translational modifications. They can be used for protein characterization by correlating experimental and theoretical MS/MS data, the latter generated from possible peptides in a protein sequence database. The characterization can include, but is not limited, to sequencing amino acids of the protein fragments, determining protein sequencing, determining protein de novo sequencing, locating post-translational modifications, or identifying post translational modifications, or comparability analysis, or combinations thereof.

As used herein, the term “database” refers to a compiled collection of protein sequences that may possibly exist in a sample, for example in the form of a file in a FASTA format. Relevant protein sequences may be derived from cDNA sequences of a species being studied. Public databases that may be used to search for relevant protein sequences included databases hosted by, for example, Uniprot or Swiss-prot. Databases may be searched using what are herein referred to as “bioinformatics tools.” Bioinformatics tools provide the capacity to search uninterpreted MS/MS spectra against all possible sequences in the database(s), and provide interpreted (annotated) MS/MS spectra as an output. Non-limiting examples of such tools are Mascot (www.matrixscience.com), Spectrum Mill (www.chem.agilent.com), PLGS (www.waters.com), PEAKS (www.bioinformaticssolutions.com), Proteinpilot (download.appliedbiosystems.com//proteinpilot), Phenyx (www.phenyx-ms.com), Sorcerer (www.sagenresearch.com), OMS SA (www.pubchem.ncbi.nlm.nih.gov/omssa/), X!Tandem (www.thegpm.org/TANDEM/), Protein Prospector (prospector.ucsfedu/prospector/mshome.htm), Byonic (www.proteinmetrics.com/products/byonic) or Sequest (fields.scripps.edu/sequest).

In some embodiments, the sample comprising the protein of interest can be treated by adding a reducing agent to the sample.

As used herein, the term “reducing” refers to the reduction of disulfide bridges in a protein. Non-limiting examples of the reducing agents used to reduce the protein are dithiothreitol (DTT), β-mercaptoethanol, Ellman's reagent, hydroxylamine hydrochloride, sodium cyanoborohydride, tris(2-carboxyethyl)phosphine hydrochloride (TCEP-HC1), or combinations thereof. In some specific embodiments, the treatment can further include alkylation. In some other specific exemplary embodiments, the treatment can include alkylation of sulfhydryl groups on a protein.

As used herein, the term “treating” or “isotopically labeling” can refer to chemical labeling a protein. Non-limiting examples of methods to chemically label a protein include Isobaric tags for relative and absolute quantitation (iTRAQ) using reagents, such as 4-plex ,6-plex, and 8-plex; reductive demethylation of amines, carbamylation of amines, ¹⁸O-labeling on the C-terminus of the protein, or any amine- or sulfhydryl- group of the protein to label amines or sulfhydryl group.

In some embodiments, the sample comprising the protein of interest can be digested prior to subjecting it to a chromatography column.

As used herein, the term “digestion” refers to hydrolysis of one or more peptide bonds of a protein. There are several approaches to carrying out digestion of a protein in a sample using an appropriate hydrolyzing agent, for example, enzymatic digestion or non-enzymatic digestion.

As used herein, the term “hydrolyzing agent” refers to any one or combination of a large number of different agents that can perform digestion of a protein. Non-limiting examples of hydrolyzing agents that can carry out enzymatic digestion include trypsin, endoproteinase Arg-C, endoproteinase Asp-N, endoproteinase Glu-C, outer membrane protease T (OmpT), immunoglobulin-degrading enzyme of Streptococcus pyogenes (IdeS), chymotrypsin, pepsin, thermolysin, papain, pronase, and protease from Aspergillus Saitoi. Non-limiting examples of hydrolyzing agents that can carry out non-enzymatic digestion include the use of high temperature, microwave, ultrasound, high pressure, infrared, solvents (non-limiting examples are ethanol and acetonitrile), immobilized enzyme digestion (IMER), magnetic particle immobilized enzymes, and on-chip immobilized enzymes. For a recent review discussing the available techniques for protein digestion see Switazar et al., “Protein Digestion: An Overview of the Available Techniques and Recent Developments” (J. Proteome Research 2013, 12, 1067-1077). One or a combination of hydrolyzing agents can cleave peptide bonds in a protein or polypeptide, in a sequence-specific manner, generating a predictable collection of shorter peptides.

The consecutive labeling of method steps as provided herein with numbers and/or letters is not meant to limit the method or any embodiments thereof to the particular indicated order.

Various publications, including patents, patent applications, published patent applications, accession numbers, technical articles and scholarly articles are cited throughout the specification. Each of these cited references is herein incorporated by reference, in its entirety and for all purposes.

The disclosure will be more fully understood by reference to the following Examples, which are provided to describe the disclosure in greater detail. They are intended to illustrate examples and should not be construed as limiting the scope of the disclosure.

### EXAMPLES

### Materials

Deionized water was provided by a Milli-Q integral water purification system installed with a MilliPak Express 20 filter (Millipore Sigma, Burlington, Mass., Cat. NO. MPGP02001). Ammonium acetate (LC/MS grade) was purchased from Sigma-Aldrich (St. Louis, Miss., Prod. No. 73594). Peptide N-glycosidase F (PNGase F) was purchased from New England Biolabs Inc (Ipswich, Mass., Prod. No. P0704L). FabRICATOR® was purchased from Genovis (Cambridge, Mass., Prod. No. AO-FR1-250). Invitrogen UltraPure 1 M Tris-HCl buffer, pH 7.5 (Ref. No. 15567-027), Pierce™ DTT (Dithiothreitol, No-Weigh™ Format, Ref. No. A39255), and Acetonitrile (ACN; Optima LC/MS grade, Prod. No. A955-4) were purchased from Thermo Fisher Scientific (Waltham, Mass.). Formic acid (FA, 98-100%, Suprapur for trace metal analysis) was purchased from Millipore Sigma (Burlington, Mass., Prod. No. 1.11670.0250). 2-propanol (IPA; HPLC grade) was purchased from Sigma Aldrich (St. Louis, Miss., Prod. No. 65-0447-4L).

### Sample Preparation

All mAbs were produced in CHO cells at Regeneron Pharmaceutical, Inc. The mAb3 enriched HMW sample was generated by fractionating the HMW species from a mAb3 DS sample using a semi-preparation scale SEC column. The final enriched HMW sample contains 0.7% trimer, 66.8% dimer and 32.5% monomer. Prior to desalting SEC-MS analysis, limited reduction was performed by treating mAbl with 2 mM DTT in 50 mM Tris-HCl (pH 7.5) at 37° C. for 30 min to only reduce inter-chain disulfide bonds. For intact level analysis, all mAb samples, including enriched HMW samples, individual DS samples, and co-formulated DP samples, were treated with PNGase F (1 IUB milliunit per 10 μg of protein) at 45° C. in 50 mM Tris-HCl (pH 7.0) for 1 hour to remove the N-glycan chains from each heavy chain CH2 domain. For subdomain analysis, an aliquot of the deglycosylated mAb3 HMW sample and mAb4 DS samples was each subjected to site-specific digestion with FabRICATOR (1 IUB milliunit per 1 μg of protein) in 50 mM Tris-HCl (pH 7.5) at 37° C. for 1 hour, to generate the F(ab)′₂ and Fc fragments.

PCD-Assisted nSEC-MS

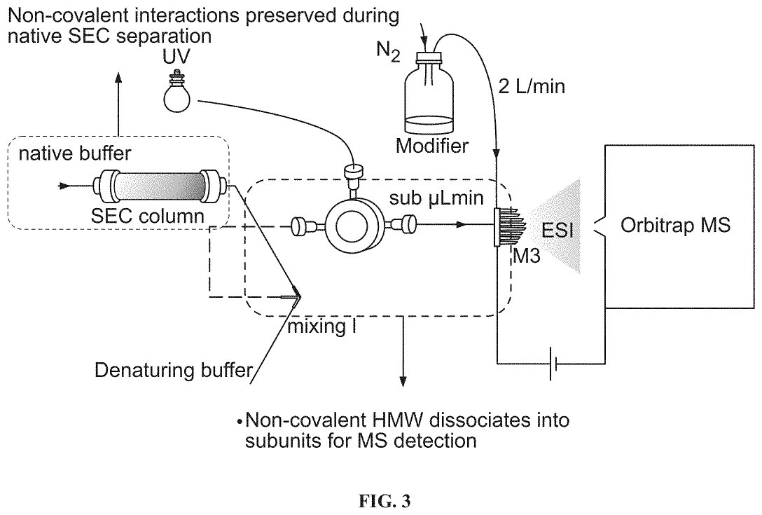

Native SEC chromatography was performed on an UltiMate 3000 UHPLC System (Thermo Fisher Scientific, Bremen, Germany) equipped with an Acquity BEH200 SEC column (4.6×300 mm, 1.7 μm, 200 Å; Waters, Milford, Mass.) with the column compartment set to 30° C. An isocratic flow of 150 mM ammonium acetate at 0.2 mL/mL was applied to separate and elute protein size variants. To enable post-column denaturation, a denaturing solution consisting of 60% ACN, 36% water, and 4% FA was delivered by a secondary pump at a flow rate of 0.2 mL/min and then mixed with the SEC eluent (1:1 mixing) using a T-mixer before subjected to MS detection. To enable online native MS analysis, the combined analytical flow (0.4 mL/min) was split into a microflow (<10 μL/min) for nano-electrospray ionization (NSI)-MS detection and a remaining high flow for UV detection (FIG. 3). A Thermo Q Exactive UHMIR (Thermo Fisher Scientific, Bremen, Germany) equipped with a Microflow-Nanospray Electrospray Ionization (MnESI) Source and a Microfabricated Monolithic Multi-nozzle (M3) emitter (Newomics, Berkley, Calif.) was used for native MS analysis. A detailed experimental setup and instrument parameters are described in Yan Y, Xing T, Wang S, Li N. Versatile, sensitive, and robust native LC-MS platform for intact mass analysis of protein drugs. J Am Soc Mass Spectrom 2020: 31(10): 2171-2179, which is incorporated by reference in its entirety. To disable PCD, the flow of the denaturing solution was set to zero. Desalting SEC-MS analysis of the partially reduced mAb 1 was performed in a similar fashion using Acquity BEH200 SEC guard column (4.6×30 mm, 1.7 μm, 200 Å).

### Data Analysis

Intact mass spectra from nSEC-MS analysis under native or PCD conditions were deconvoluted using Intact Mass™ software from Protein Metrics.

### Example 1

### PCD-Assisted nSEC-MS Method

To improve nSEC-MS-based characterization of mAb HMW species, a post-column denaturation (PCD) strategy is introduced to dissociate non-covalent HMW complexes after SEC separation before MS detection. This strategy is highly desirable, as it not only enables improved assignment of non-covalent HMW complexes by confirming the constituent subunits, but also provides more accurate mass measurement of non-dissociable HMW species by reducing the interference from co-eluting, non-covalent species.

Taking advantage of a previously described nLC-MS platform (Yan et al. (2020), supra) that can accommodate a high flow rate (up to 0.8 mL/min), integration of PCD with nSEC-MS can be readily achieved by introducing a post-column denaturant flow (0.2 mL/min) to the nSEC flow (0.2 mL/min) via a T-mixer (FIG. 3).

The denaturing solvent was carefully selected based on two primary considerations. First, the final flow after post-column mixing should still be highly compatible with direct MS detection. Second, because of the short denaturation time (e.g., less than 1 second from the T-mixer to MS), the desired denaturing solvent should be capable of disrupting the majority of the non-covalent interactions instantaneously after post-column mixing.

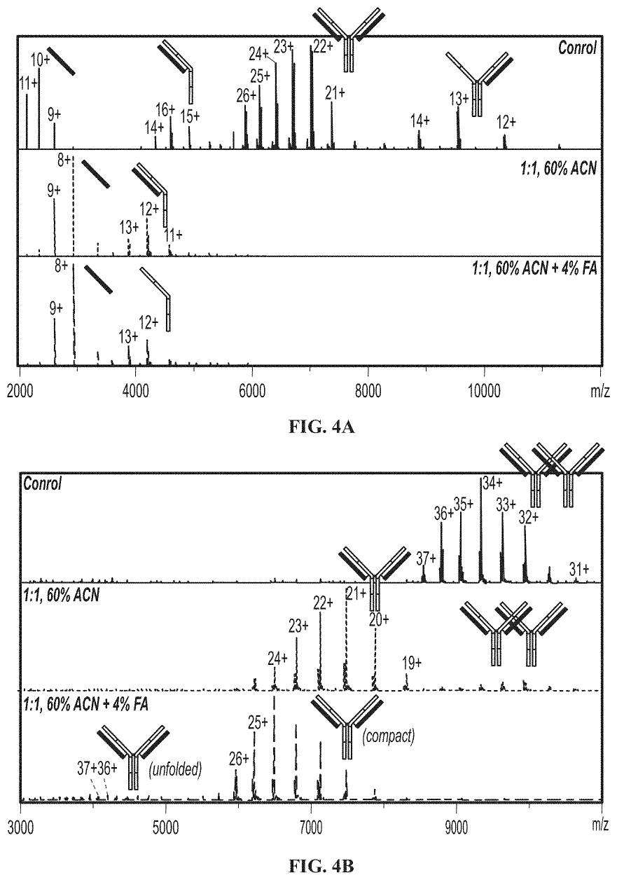

After evaluating a series of denaturing solvent systems containing varying levels of acetonitrile (ACN) and formic acid (FA), an optimized formula comprised of 60% ACN, 4% FA, and 36% water was selected for PCD application. To assess the effectiveness of the selected denaturing solvent, mAb 1 (IgG4 subclass) was partially reduced (inter-chain disulfide bonds disrupted) and subjected to PCD-assisted nSEC-MS analysis using a short SEC guard column (FIG. 4A). Because of the strong inter-chain non-covalent interactions between the two CH3 domains and between the N-terminal regions of heavy and light chains (HC and LC) in IgG4 molecules (Rose R J, Labrijn A F, van den Bremer E T, Loverix S, Lasters I, van Berkel P H, van de Winkel J G, Schuurman J, Parren P W, Heck A J. Quantitative analysis of the interaction strength and dynamics of human igg4 half molecules by native mass spectrometry. Structure 2011: 19(9): 1274-1282), the partially reduced mAbl was predominantly detected as an intact H2L2 complex under nSEC-MS conditions. Only low levels of HL, H2L and LC species were observed, which were likely generated via in-source dissociation (FIG. 4A, black trace). In contrast, after applying PCD conditions (60% ACN/4% FA), these non-covalent complexes (e.g., H2L2, H2L, and HL) were completely dissociated and detected as free HC and LC (FIG. 4A, red trace). An alternative denaturing solvent containing only 60% ACN was also tested, which showed comparable effectiveness in dissociating the partially reduced mAb complex (FIG. 2A, orange trace).

In another example, the mAb2 dimer species detected by nSEC-MS analysis (FIG. 4B, black trace) displayed a near-complete dissociation into monomers upon application of PCD (60% ACN/4% FA) (FIG. 4B, red trace), suggesting the majority, if not all, of the dimer species were non-covalent. In addition, low levels of highly charged monomer signal, corresponding to the unfolded species, were also observed in the low m/z region. Unlike the first example, application of the alternative denaturing solvent containing 60% ACN alone did not lead to complete dissociation of the mAb2 dimer species (FIG. 4B, orange trace), suggesting the combination of low pH and organic solvent is more effective in disrupting the non-covalent interactions. Subsequently, the developed PCD conditions have also been applied to other non-covalent systems (e.g., antibody-antigen complexes and virus capsids), where rapid and effective dissociation could always be achieved (data not shown). Therefore, the developed PCD conditions are considered effective in disrupting the majority of non-covalent interactions present in mAb HMW complexes, although it is still possible that some tightly associated non-covalent complexes may survive the treatment. Lastly, it is important to note that applying the reported nLC-MS platform (Yan et al. (2013), supra), the mAb-related species all exhibited “native-like” mass spectra under the selected PCD conditions (60% ACN/4% FA). This feature is highly desirable, as it reduces the spectral overlapping from multiple species that are simultaneously dissociated from the same complexes and detected in the same MS scan. For example, under PCD conditions, the MS signal of the dissociated HC and LC were well isolated on the m/z scale with minimal overlapping (FIG. 4A). In addition, compared to typical ESI-MS spectra under denaturing conditions, “native-like” spectra exhibit much fewer charge states and greater spatial resolution, making them easier to be interpreted and processed (e.g., generating extracted ion chromatograms).

### Example 2

### PCD-Assisted nSEC-MS Analysis of Enriched HMW Species

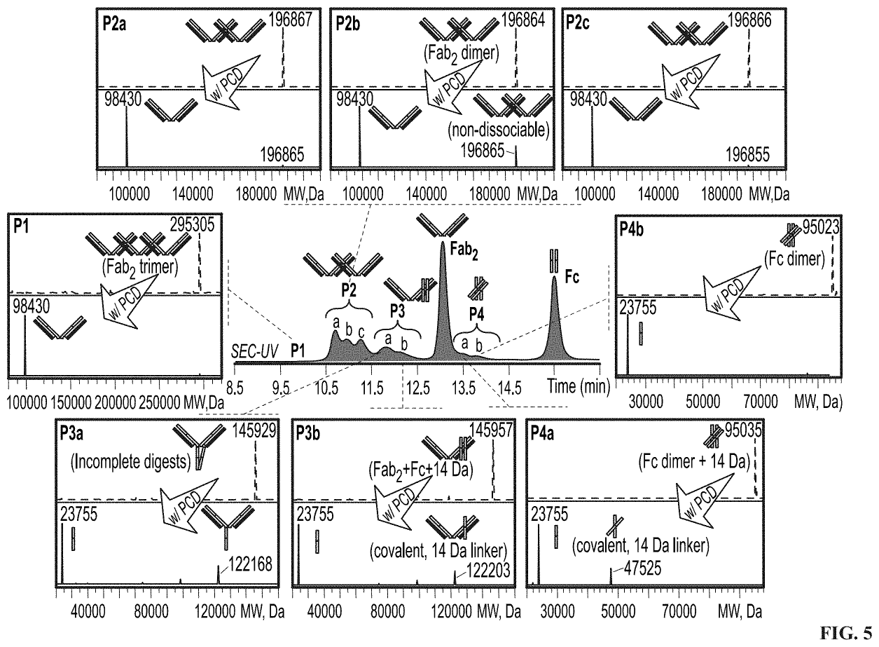

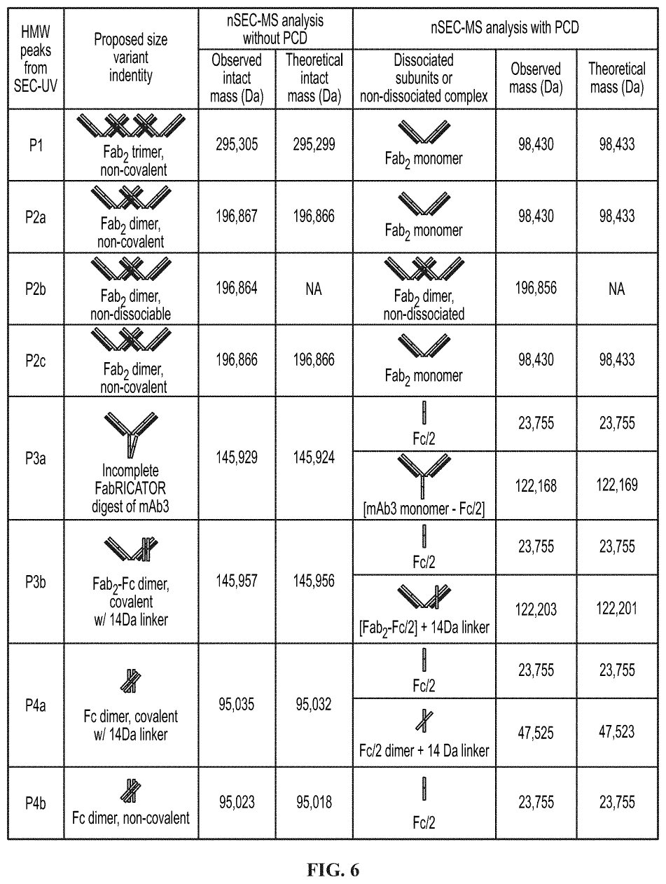

Extended characterization of mAb HMW species is often required at the late stage of program development, as part of the DS heterogeneity characterization. Limited enzymatic digestion (e.g., IdeS digestion) followed by intact mass analysis is frequently performed on the enriched BMW material to understand the interaction interfaces at subdomain levels. For this purpose, a mAb3 enriched HMW sample mainly containing dimeric species was treated with IdeS digestion before subjected to PCD-assisted nSEC-MS analysis. As IdeS cleaves the mAb molecule under the hinge region releasing F(ab)′₂ and Fc fragments, this strategy allows effective characterization of the dimeric interactions at subdomain levels. SEC-UV analysis of the digested HMW sample (FIG. 5, middle) exhibited multiple resolved UV peaks, including two major ones corresponding to F(ab)′₂ and Fc monomers, and four other peaks (P1-P4) likely corresponding to HMW-related species as a result of various subdomain interactions present in the enriched BMW sample. Subsequently, the accurate mass measurement from nSEC-MS analysis was used to assign the identity of each peak (FIG. 5, FIG. 6). Although intact mass measurement of the native complexes can readily differentiate aggregation states and interacting partners (e.g., F(ab)′₂ trimer in P1, F(ab)′₂ dimer in P2, F(ab)′₂-Fc heterodimer in P3, and Fc dimer in P4), detailed elucidation of each species was still challenging due to a considerable amount of ambiguity from intact mass-based assignments.

For example, the Fc dimer in P4b exhibited an observed mass (95,023 Da) consistent with the predicted mass of a non-covalent dimer (95,018 Da), while the Fc dimer in P4a exhibited a mass increase of approximately 14 Da (FIG. 5) compared to the predicted mass. This mass increase can be potentially attributed to the presence of either an oxidation modification (+16 Da) within a non-covalent complex or a covalent crosslink (e.g., 14 Da between two histidine residues) maintaining a covalent complex. Unfortunately, the mass resolution and accuracy achieved at the intact complex level cannot lead to an unambiguous assignment and differentiate the two very different scenarios. Similarly, confident elucidation of F(ab)′₂-Fc heterodimer in P3 was also compounded by the possible co-existence of both non-covalent and covalent dimer species, as well as incomplete reaction products from IdeS digestion, all of which would only exhibit small mass differences between each other. Finally, although nSEC-MS analysis readily confirmed that the three partially resolved peaks, P2a, P2b, and P2c, all contained F(ab)′₂ dimer species with similar observed masses (196,864-196,867 Da), no other meaningful information can be retrieved from this analysis to characterize the apparently heterogeneous F(ab)′₂- F(ab)′₂ interactions present in the HMW sample.

To reduce the ambiguities and improve the characterization, PCD was implemented post-SEC separation to provide a second dimension of separation based on interaction nature. For example, under PCD conditions, distinctive dissociation behaviors were observed for the Fc dimer species in P4a and P4b. The Fc dimer in P4b, which had already been tentatively assigned as a non-covalent species based on the observed mass of the native complex, underwent a complete dissociation into Fc/2 subunits under PCD conditions. This result confirmed the non-covalent nature of the Fc dimer in P4b. In contrast, application of PCD in P4a led to the formation of both Fc/2 subunits (e.g., dissociated from the non-covalent Fc complex) and a non-dissociable Fc/2 dimer species. Consistent with the larger mass of the Fc dimer in P4a as detected under native conditions, the non-dissociable Fc/2 dimer also showed a mass increase of approximately 14 Da comparing to that of a non-covalent Fc/2 dimer. This delta mass was proposed to correspond to a previously reported covalent crosslink that occurs between two histidine (His) residues (crosslinker mass: 13.98 Da) (Xu CF, Chen Y, Yi L, Brantley T, Stanley B, Sosic Z, Zang L. Discovery and characterization of histidine oxidation initiated cross-links in an iggl monoclonal antibody. Anal Chem 2017: 89(15): 7915-7923; Powell T, Knight MJ, Wood A, O'Hara J, Burkitt W. Photoinduced cross-linking of formulation buffer amino acids to monoclonal antibodies. Eur J Pharm Biopharm 2021: 160(35-41)). Subsequent peptide mapping analysis also identified several His-His crosslinked dipeptides from the Fc region that likely contributed to the Fc dimer in P4a (data not shown). The same covalent crosslink was also observed for the F(ab)′₂-Fc dimer in P3b, which was measured ˜14 Da higher in mass comparing to that of a non-covalent F(ab)′₂-Fc dimer. Application of PCD further confirmed this assignment by dissociating this species into a Fc/2 subunit and a non-dissociable F(ab)′₂-Fc/2 complex that also exhibited a mass increase of approximately 14 Da due to the His-His crosslink.

In contrast, the species in P3a displayed an observed mass approximately 18 Da lower than that of a non-covalent F(ab)′₂-Fc dimer and was readily dissociated into a Fc/2 and a complementary, Fc/2-clipped mAb species under PCD conditions. Therefore, the species in P3a was assigned as an incomplete IdeS digestion product with only one heavy chain cleaved. Finally, despite the similar observed masses at the intact complex level, the F(ab)′₂ dimer in P2b exhibited different dissociation behavior than those in P2a and P2c under PCD conditions (FIG. 5). Specifically, with PCD applied, the dimer species in P2a and P2c underwent near-complete dissociation, leading to the detection of solely F(ab)′₂ monomers. This observation indicated the non-covalent nature of the F(ab)′₂ dimers in both P2a and P2c, which were separated by SEC likely due to conformational differences. In contrast, P2b showed a significant amount of F(ab)′₂ dimer remained non-dissociable under PCD conditions, suggesting the presence of a “covalent-like” F(ab)′₂ dimer. Additionally, as the co-eluting, non-covalent F(ab)′₂ dimer was dissociated, a more accurate mass measurement of the non-dissociable dimer in P2b could be achieved. Indeed, this analysis revealed that the non-dissociable dimer in P2b exhibited a lower mass (196,856 Da) than that of a non-covalent dimer (theoretical mass: 196,865 Da), suggesting the possible presence of a covalent crosslink with a negative delta mass. Although identification of this covalent crosslink is still ongoing and outside the scope of this manuscript, the information from the PCD-assisted nSEC-MS analysis is valuable to guide the investigation.

### Example 3

### PCD-Assisted nSEC-MS Analysis of HMW Species in Unfractionated DS Samples

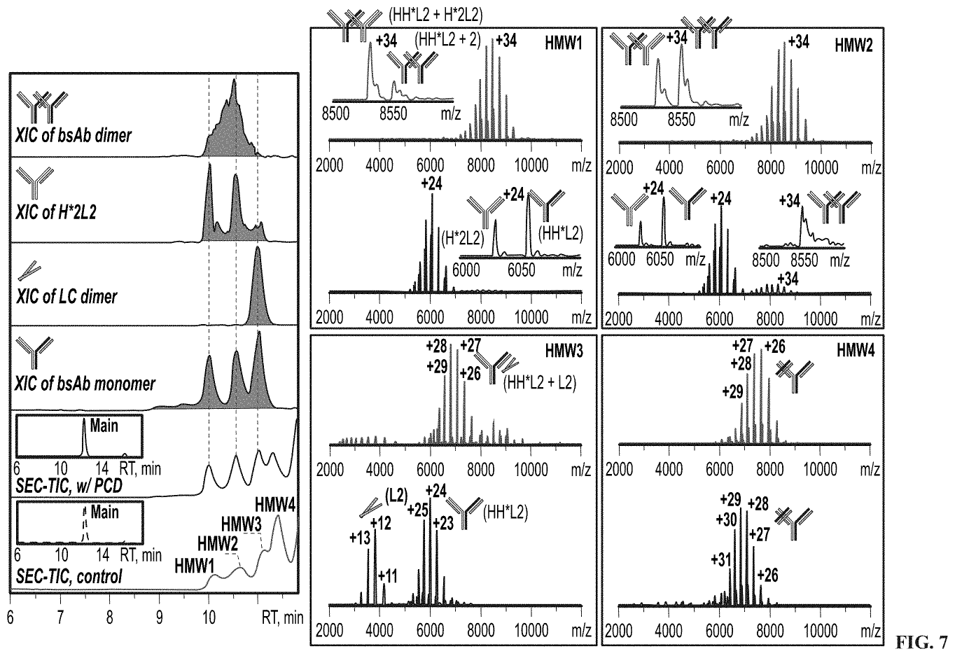

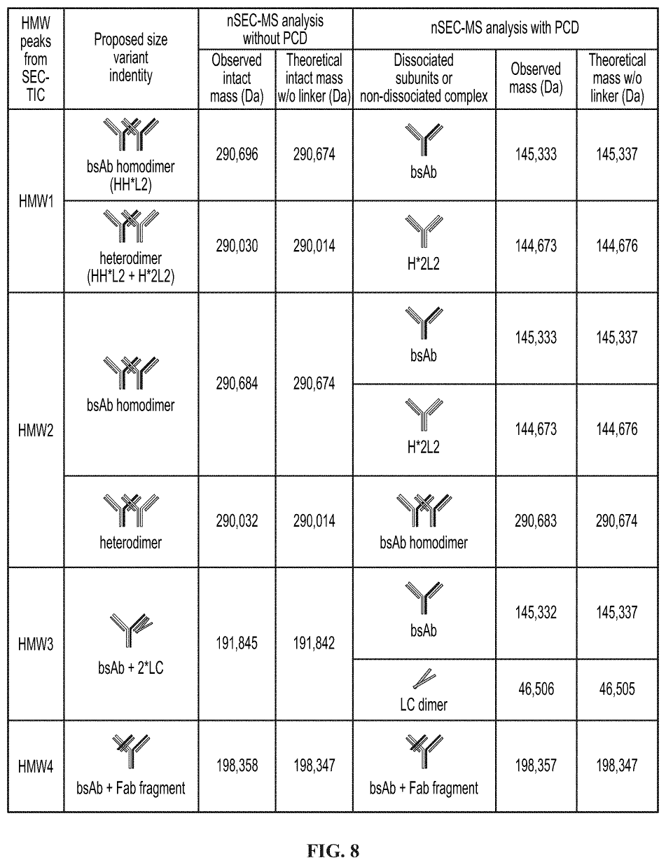

Direct analysis of HMW species from unfractionated mAb DS samples is highly desirable, as it is less resource-demanding and eliminates potential changes in the HMW profile (e.g., artificial HMW formation or dissociation of labile HMW species) due to sample handling. To demonstrate the applicability of the PCD-assisted nSEC-MS method in elucidating complex HMW species from unfractionated samples, a bispecific antibody (bsAb) DS sample, which exhibited a complicated HMW profile (four partially-resolved HMW peaks) during SEC separation, was subjected to the analysis (FIG. 7). Consisting of two identical light chains (LC) and two different heavy chains (HC and HC*), the bsAb (HH*L2) DS samples often contain low levels of monospecific mAb impurities (H2L2 and H*2L2) that can further contribute to the increased complexity of the HMW species. For example, nSEC-MS analysis indicated the presence of two different dimers in both HMW1 and HMW2 peaks, including a bsAb homodimer (HH*L2×2) and a heterodimer (HH*L2+H*2L2) consisting of a bsAb and a monospecific H*2L2 species (deconvoluted mass shown in FIG. 8). The relative abundance of the heterodimer species is slightly higher in HMW1 peak compared to HMW2 peak. Application of PCD further supported these assignments, where both bsAb and H*2L2 monomers were dissociated from the dimer species and detected in HMW1 and HMW2 peaks. Interestingly, application of PCD resulted in a complete dissociation of the heterodimers in both HMW1 and HMW2 peaks, indicating the non-covalent nature of these species. In contrast, the bsAb homodimers in HMW2 peak underwent a partial dissociation while a noticeable amount remained intact under PCD conditions, suggesting the presence of the non-dissociable bsAb homodimer. As the monomer species can only be generated from the dissociation of the non-covalent dimers under PCD conditions, the extracted ion chromatograms (XICs) constructed using the monomer signal (e.g., bsAb monomer and H*2L2 monomer) could represent the elution profiles of the non-covalent dimers.

Meanwhile, the XIC constructed using the bsAb homodimer signal under PCD conditions could represent the elution profile of the non-dissociable bsAb homodimers. Applying this strategy, it was clear that both the non-covalent homodimer and the non-covalent heterodimer eluted in HMW1 and HMW2 peaks, while the non-dissociable bsAb homodimer eluted in a broad and distinctive region with the peak apex aligned with HMW2 peak (FIG. 7, left panel). Similarly, based on accurate mass measurement, the nSEC-MS analysis tentatively identified the HMW3 peak as a complex comprised of a bsAb monomer and two extra LCs. Subsequently, application of PCD not only confirmed the proposed composition, but also revealed that the two extra LCs were present as a non-dissociable dimer (e.g., likely via inter-chain disulfide bond) and then associated with a bsAb molecule via non-covalent interactions. The XICs of the dissociated LC dimer and the bsAb monomer also confirmed their co-elution with HMW3 peak, further supporting this assignment. Lastly, based on accurate mass measurement, the species in HMW4 peak was proposed to be a complex consisting of a bsAb monomer and a Fab fragment due to a clipping in CH2 domain. As this species remained intact under PCD conditions, we think that it was a degradation product resulting from the truncation of the non-dissociable bsAb homodimer species.

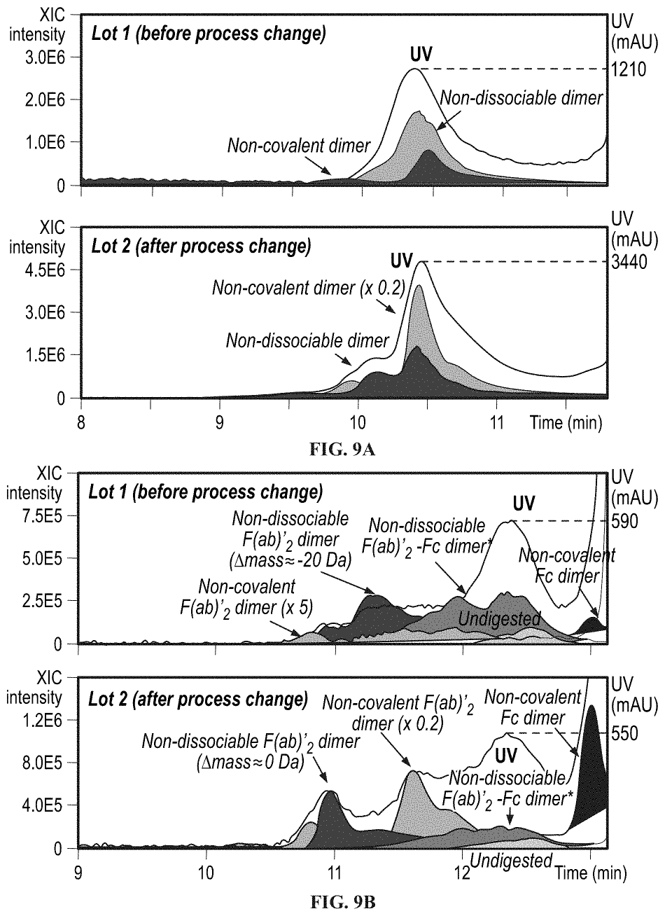

The ability to elucidate the HMW species directly from unfractionated DS samples makes the PCD-assisted nSEC-MS method ideally suited for process development support, where fast turn-around is desired to facilitate decision-making. To test the utility in this area, the method was then applied to assess the comparability of the HMW profile of a mAb program before and after process changes. As demonstrated in the SEC-UV traces, the HMW profiles of mAb4 DS lots before and after the process changes were generally comparable with minor differences in peak shape (FIG. 9A, black trace). With accurate mass measurement from the nSEC-MS analysis, the predominant HMW peaks in both lot 1 and lot 2 were readily identified as mAb4 dimer species (FIG. 9A, black trace).

Application of PCD then revealed the presence of both the non-covalent dimer (FIG. 9A, magenta trace, represented by the XIC of mAb4 monomer signal under PCD conditions) and the non-dissociable dimer (FIG. 9A, blue trace, represented by the XIC of non-dissociated mAb4 dimer signal under PCD conditions) in both DS lots. It is clear that, although the HMW species were considered generally comparable based on UV peaks and the observed masses, the distributions of the non-covalent dimer and the non-dissociable dimer were largely different between the two lots. Specifically, lot 2 contained a significantly higher level of the non-covalent dimer species, while lot 1 contained a notably higher level of the non-dissociable dimer species. Moreover, the relative abundance of the non-covalent dimer within the total HMW species can also be estimated based on the UV peak areas and the XICs generated from the PCD-assisted nSEC-UV/MS analysis using the following equation:

$$\frac{Non - covalent dimer}{Total dimer} \% = \frac{{XIC}_{D i m e r} / {XIC}_{M o n o m e r}}{{UU}_{D i m e r} / {UV}_{M o n o m e r}} , \qquad ( 1 )$$

Where XIC_{Dimer} and XIC_{Monomer} represent the integrated XIC peak areas of the monomer signal appearing in the dimer elution and monomer elution regions, respectively; UV_{Dimer} and UV_{Monomer} represent the integrated UV peak areas of the dimer and monomer peaks, respectively. In this calculation, the non-covalent dimer is quantified using the PCD-induced monomer signal in the dimer elution region and normalized against the real monomer signal. As only the monomer signal was used, discrepancy in MS responses of different species (e.g., dimer vs monomer) can be mitigated, leading to more reliable quantitation.

Using this strategy, the relative abundances of the non-covalent dimer within the total HMW species were estimated at ˜11% and ˜86% in lot 1 and lot 2 DS samples, respectively. Additionally, the non-dissociable dimers in lot 1 and lot 2 samples also exhibited different elution profiles, where lot 2 showed a higher level of the early-eluting species. (FIG. 9A, blue trace). Consistently, further analysis of the dimer interactions at the subdomain level (e.g., after IdeS digestion) (FIG. 9B) also revealed higher levels of non-covalent complexes in lot 2 DS sample, including the non-covalent F(ab)′₂ dimer (FIG. 9B, magenta trace, represented by XIC of dissociated F(ab)′₂ monomer) and the non-covalent Fc dimer (FIG. 9B, brown trace, represented by XIC of dissociated Fc/2 monomer). The non-dissociable complexes including the non-dissociable F(ab)′₂ dimer (FIG. 9B, blue trace, represented by XIC of non-dissociated F(ab)′₂ dimer) and the non-dissociable F(ab)′₂-Fc heterodimer (FIG. 9B, orange trace, represented by XIC of non-dissociated F(ab)′₂-Fc dimer) were also detected in both lots. In particular, the non-dissociable F(ab)′₂ dimer displayed a similar elution profile (FIG. 9B, blue trace) as observed at the intact level (FIG. 9A, blue trace), showing two partially separated peaks in both lots. Consistently, compared to lot 1, lot 2 showed a much higher level of the early-eluting, non-dissociable F(ab)′₂ dimer species (FIG. 9A and 5B, blue trace). Subsequently, accurate mass measurement of the non-dissociable complexes was achieved by removing the interference from the non-covalent complexes under PCD conditions and was then used to study the nature of interactions.

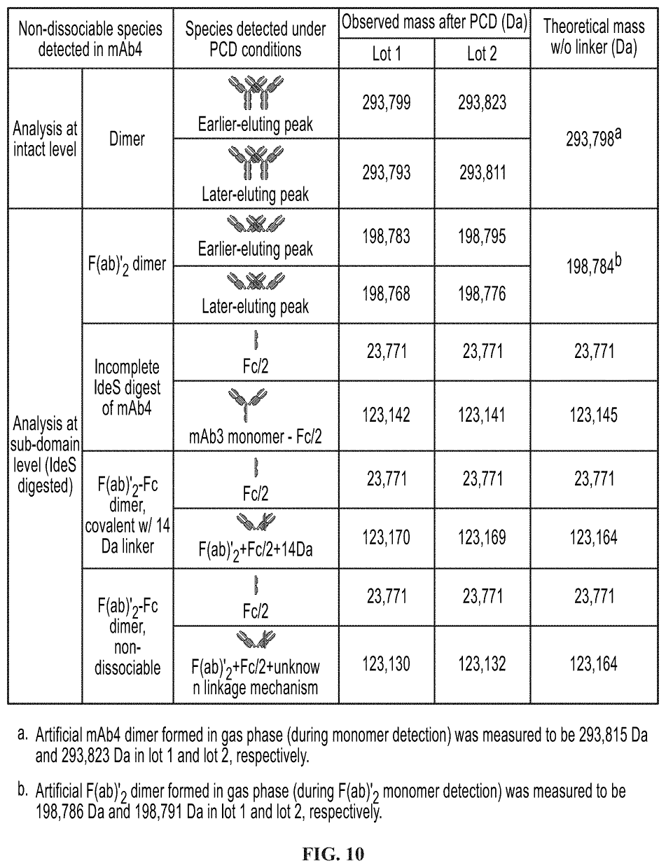

It is observed that, compared to the predicted mass of a non-covalent F(ab)′₂ dimer, the late-eluting, non-dissociable F(ab)′₂ dimer consistently exhibited a mass decrease of approximately 20 Da, suggesting the potential presence of a covalent crosslink with a negative delta mass. In contrast, the observed mass of the early-eluting, non-dissociable F(ab)′₂ dimer was comparable to that of a non-covalent dimer, suggesting they were formed either via a small covalent crosslink or through a strong non-covalent interaction that was maintained under PCD conditions. Lastly, the non-dissociable F(ab)′₂-Fc heterodimers in both lot 1 and lot 2 exhibited a broad elution profile (FIG. 9b, orange trace), which was attributed to three different species including 1) a [F(ab)′₂-Fc]+14 Da covalent dimer likely formed via a His-His crosslink; 2) a [F(ab)′₂-Fc]-30 Da covalent dimer with an unknown crosslink, and 3) a [F(ab)′₂-Fc]-18 Da complex due to incomplete IdeS digestion (Fc/2-clipped mAb) (FIG. 10). Together, the differences in HMW profile between the two DS lots as a result of process changes can be examined in great detail and attributed to differences at subdomain level interactions. Although a complete understanding of these interactions (the exact covalent crosslinks in particular) might still require offline fractionation and further characterization, the rapid analysis of the unfractionated DS samples provided necessary information to assess the impact from process changes and build a framework for risk assessment.

### Example 4

### PCD-Assisted nSEC-MS Analysis of Hetero-Intermolecular Interactions in Co-Formulated mAb Samples

Characterization of the HMW species formed in co-formulated mAb drug product (DP) samples (e.g., containing more than one therapeutic mAbs) under storage or stability conditions is important over the course of development (Guidance for industry: Codevelopment of two or more new investigational drugs for use in combination. Center for drug evaluation and research. Rockville (MD): US Food and Drug Administration 2013). Such analysis, however, presents unique analytical challenges due to the highly complex HMW profiles frequently present in these samples involving both the homo- and hetero-intermolecular interactions (Kim J, Kim Y J, Cao M, De Mel N, Albarghouthi M, Miller K, Bee J S, Wang J, Wang X. Analytical characterization of coformulated antibodies as combination therapy. MAbs 2020: 12(1): 1738691).

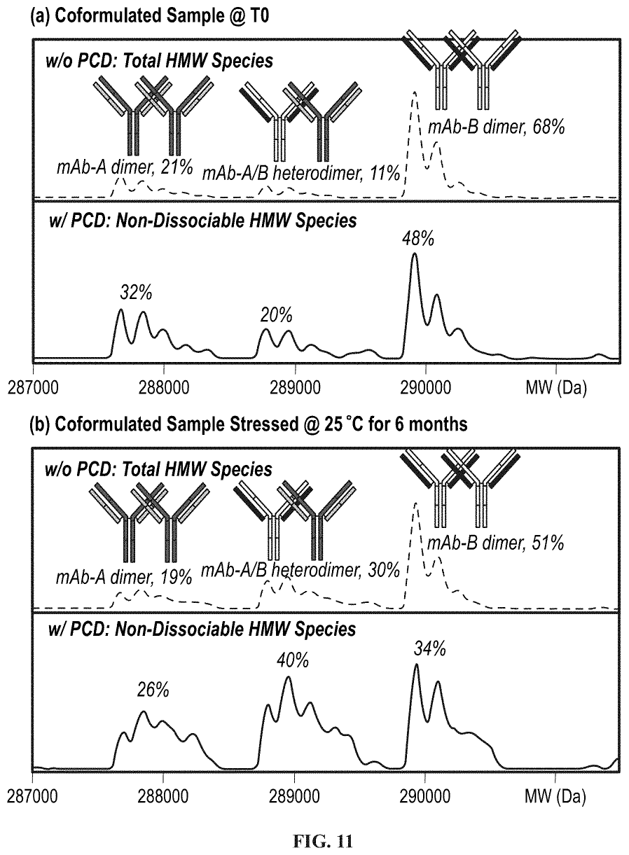

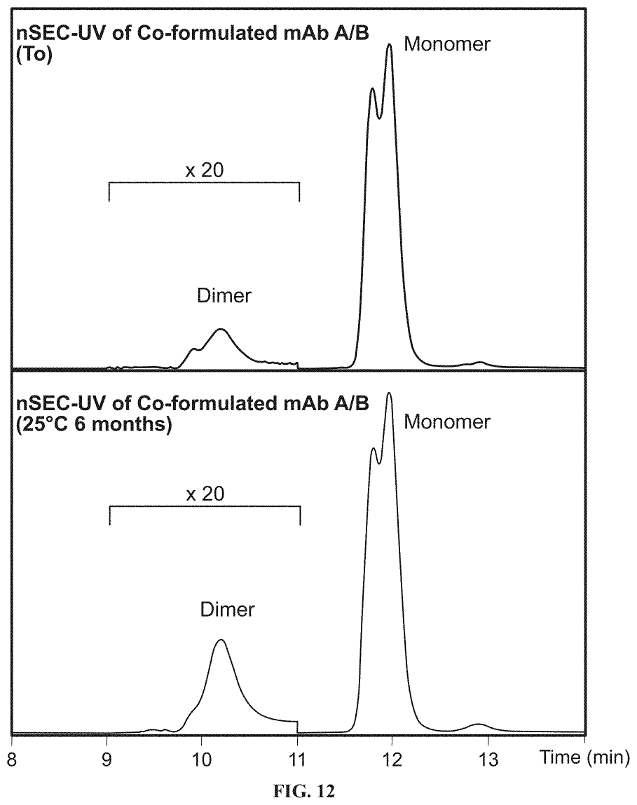

To tackle these challenges, the utility of the PCD-assisted nSEC-MS method was also evaluated in studies to support the development of co-formulated mAb programs. As an example, a co-formulated DP sample consisting of two mAbs (mAb-A and mAb-B) were tested under accelerated stability conditions. Three major HMW species, namely, mAb-A homodimer, mAb-B homodimer, and mAb-AB heterodimer, were readily identified in both T0 (unstressed, total HMW%=0.7%) and T6m (25° C. for 6 months, total HMW% =1.5%) samples by nSEC-MS analysis based on their different molecular weights (FIG. 11, FIG. 12). Using the integrated peak areas from the deconvoluted mass spectra, the relative abundances of the three dimers could be estimated. Interestingly, in addition to the two homodimers, a low but noticeable level of the mAb-AB heterodimer was readily detected in the TO sample (FIG. 11A), suggesting the hetero-intermolecular interaction was likely initiated spontaneously when the two mAbs were mixed. After being stored at 25° C. for 6 months, the relative abundance of the mAb-AB heterodimer increased significantly (from 11% to 30%), while the abundances of the mAb-A homodimer and the mAb-B homodimer remained unchanged or decreased, respectively.

This observation suggested that the mAb-AB heterodimer grew at a faster rate than the homodimers under the accelerated stability conditions. In addition, with the application of PCD, the same calculation and comparison can be made for the non-dissociable dimer species (FIG. 11, red trace), providing a high-level assessment of the interaction nature. For example, under PCD conditions, a notable decrease in relative abundance was observed for the mAb-B homodimer in both T0 and T6m samples, suggesting the non-covalent interaction contributed more significantly to the formation of mAb-B homodimer than the other two species (e.g., mAb-A homodimer and mAb-AB heterodimer). In addition, it was observed that the non-dissociable mAb-AB heterodimer exhibited a faster growth rate (from 20% to 40%) and became the most abundant non-dissociable dimer species after 6 months. This rapid analysis showed that the hetero-intermolecular interaction between mAb-A and mAb-B was favourable under accelerated stability conditions, likely via covalent crosslinks or tight but non-covalent interactions. This information was valuable to guide the future investigation to elucidate the exact interactions responsible for the hetero-dimerization, and therefore, facilitate the formulation development to minimize this type of interaction.

Comprehensive characterization of the BMW size variants is highly important during the development of therapeutic mAbs. The development of a PCD-assisted nSEC-MS method of the present invention enables efficient dissociation of the non-covalent BMW complexes for improved MS characterization. Specifically, application of PCD not only allows differential detection but also improves identification of both non-covalent and non-dissociable BMW species. By confirming the constituent subunits, the identification of large and unexpected non-covalent HMW complexes can be achieved with greater confidence. By removing the interference from the co-eluting, non-covalent species, more accurate mass measurement of the non-dissociable BMW complexes can be obtained, and therefore, facilitate the identification of the potential crosslinks.

Furthermore, using this method, the elution profile of each HMW complex can be readily reconstructed using XICs of either the intact ensemble (for non-dissociable species) or the constituent subunits (for non-covalent species), which adds further confidence to the identification. Due to the excellent sensitivity and specificity, this method is highly effective in elucidating the complex BMW species directly from the unfractionated DS samples, making it ideally suited for tasks requiring fast turn-around. Furthermore, the utility of this method was demonstrated in different applications, including in-depth HMW characterization at late stage development, comparability assessments, and for forced degradation studies. Lastly, with the growing complexity of mAb therapeutic formats (e.g., bsAb and co-formulation), this method is a valuable addition to our analytical arsenal to take on the increasing challenges associated with HMW characterization.

What is claimed is:

1. A method for characterizing at least one high molecular weight species of a protein of interest, said method comprising:
a. obtaining a sample including said protein of interest and said at least one high molecular weight species;
b. contacting said sample to a size exclusion chromatography column;
c. washing said column to collect an eluate;
d. adding a denaturing solution to the eluate to form a mixture; and
e. subjecting said mixture to a mass spectrometer to characterize said at least one high molecular weight species.

2. The method of claim 1, wherein the protein of interest is an antibody.

3. The method of claim 1, wherein said eluate includes said at least one high molecular weight species.

4. The method of claim 1, wherein said mixture of (d) is also subjected to ultraviolet detection.

5. The method of claim 1, wherein said mass spectrometer is a nano-electrospray ionization mass spectrometer.

6. The method of claim 1, wherein said mass spectrometer is operated under native conditions.

7. The method of claim 6 further comprising comparing at least one peak from a mass spectra obtained using (e) with a mass spectra obtained by carrying out an online size-exclusion chromatography-mass spectrometry of said sample of (a) under native conditions.

8. The method of claim 1, wherein said denaturing solution comprises acetonitrile, formic acid, or combination of acetonitrile and formic acid.

9. The method of claim 7, wherein said denaturing solution comprises about 60% v/v acetonitrile and 4% v/v formic acid.

10. The method of claim 7, wherein said denaturing solution comprises about 60% v/v acetonitrile.

11. The method of claim 7, wherein said mass spectrometer is operated under native conditions.

12. The method of claim 1, wherein said sample of (a) is digested using a hydrolyzing agent prior to (b).

13. The method of claim 11, wherein said hydrolyzing agent is a protease enzyme.

14. The method of claim 11, wherein said protease enzyme is IdeS.

15. The method of claim 1, wherein a flow of said mixture of (d) in said mass spectrometer is less than about 10 μL/min.

16. The method of claim 1, wherein said mixture of (d) split into said mass spectrometer and ultraviolet detector.

17. The method of claim 1, wherein a desolvation gas is added to said mixture of (d) prior to subjecting it to mass spectrometer.

18. The method of claim 16, wherein a multi-nozzle emitter is used to add said desolvation gas with said mixture of (d).

19. The method of claim 1, wherein at least one high molecular weight species is a non-covalent high molecular weight species of said protein of interest.

20. The method of claim 1, wherein at least one high molecular weight species is a no-dissociable high molecular weight species of said protein of interest.

21. The method of claim 1 further comprising comparing at least one peak from a mass spectra obtained using (e) with a mass spectra obtained by carrying out an online size-exclusion chromatography-mass spectrometry of said sample of (a).
